(19) United States
(12) Patent Application Publication
(10) Pub. No.: US 2004/0049253 A1
(43) Pub. Date: Mar. 11, 2004

(54) Therapeutic wrap

(76) Inventor: Terry Bryant, Lake Forest, CA (US)

Correspondence Address: LEONARD TACHNER,, A PROFESSIONAL LAW CORPORATION, SUITE 38-E, 17961 SKY PARK CIRCLE, IRVINE, CA, 92614-6364, US

(21) Appl. No.: 10/241,611

(22) Filed: Sep. 10, 2002

(51) Int. Cl. A61F 7/00

(52) U.S. Cl. 607/108; 607/112

(57) ABSTRACT

A terry cloth fabric member is shaped to hold a large quantity of ice cubes or a heated pack in contact with a limb or joint. It's adjustability is derived from expandable material in margins formed along opposing edges and from a plurality of Velcro fastening strips at one end of the wrap and extending between the expandable margins. The fabric is configured to have a slightly concave interior to receive ice cubes and hold the ice cubes or other cooling medium in contact with a joint or limb without forceful engagement which could otherwise exacerbate the injury. The expandable margins gently grip the limb or joint area, forming a seal-like engagement to minimize leakage as the ice melts.

### BACKGROUND OF THE INVENTION

1. Field of the Invention

The present invention relates generally to a fabric-based therapeutic wrap and more specifically to a low cost wrap shaped to hold ice cubes or a heated pack against a part of a limb or joint in a self-adhering, flexible configuration.

2. Background Art

There have been numerous disclosures of therapeutic wraps for use with ice or cold packs for applying a cold surface to a limb or joint. By way of example, Hill et al in U.S. Pat. No. 5,148,804 disclose an adjustable wrap that has two pockets for holding ice and a joint in alignment. Daniel in U.S. Pat. No. 5,000,176 discloses a therapeutic wrap that has a plurality of flaps for enclosing a pack around a body member. Whitehead et al in U.S. Pat. No. 5,065,758 discloses a cold pack having parallel pockets for receiving plastic blue ice cylinders. Abare in U.S. Pat. No. 4,527,566 discloses a flat wrap with pockets.

One of the disadvantages of such prior art therapeutic wraps is their relative complexity which engenders a labor intensive, high cost product. Moreover, flat wraps with pockets become cumbersome and bulky when those pockets are filled with ice cubes. Such bulky ice-filled pockets can be uncomfortable when pressed forcefully against a limb or joint that is already sensitive from an injury. Therefore, it would be highly advantageous to provide a therapeutic wrap that is simple in structure, less costly to manufacture and yet provides a more comfortable receptacle for ice cubes or the like.

### SUMMARY OF THE INVENTION

The present invention meets the aforementioned criteria for a simple, low cost wrap which is, nevertheless, easy to use, comfortable and adjustable. In a preferred embodiment, the inventive wrap comprises a terry cloth fabric member which is shaped to hold a large quantity of ice cubes in contact with a limb or joint. It's adjustability is derived from expandable material in margins formed along opposing edges and from a plurality of Velcro fastening strips at one end of the wrap and extending between the expandable margins. The fabric is configured to have a slightly concave interior to receive ice cubes and hold the ice cubes or other cooling or heating medium in contact with a joint or limb without forceful engagement which could otherwise exacerbate the injury. The expandable margins gently grip the limb or joint area, forming a seal-like engagement to minimize leakage as the ice melts. The preferred fabric, i.e., terry cloth, is highly absorbent and also minimizes leakage from the wrap.

### BRIEF DESCRIPTION OF THE DRAWINGS

The aforementioned objects and advantages of the present invention, as well as additional objects and advantages thereof, will be more fully understood herein after as a result of a detailed description of a preferred embodiment when taken in conjunction with the following drawings in which:

FIG. 1 is a three-dimensional view of a preferred embodiment shown in a closed or wrapped configuration;

FIG. 2 is a view similar to that of FIG. 1, but showing a partially unwrapped condition;

FIG. 3 is a planar view of the interior surface of the preferred embodiment;

FIG. 4 is a planar view of the exterior surface thereof;

FIG. 5 is a three-dimensional view of the interior surface ready to receive ice cubes therein;

FIG. 6 is a three-dimensional view of the interior surface with ice cubes therein;

FIG. 7 illustrates the preferred embodiment with the ice cube receiving surface it its stretched condition for receiving ice cubes prior to application to a joint or limb;

FIG. 8 is a cross-sectional view taken along lines 8-8 of FIG. 7;

FIGS. 9 and 10 are end view of the preferred embodiment shown in two different adjusted positions for small and large limbs, respectively;

FIG. 11 illustrates a small limb use of the preferred embodiment for which the FIG. 9 adjusted position is suitable; and

FIG. 12 illustrates a large limb use of the preferred embodiment for which the FIG. 10 adjusted position is suitable.

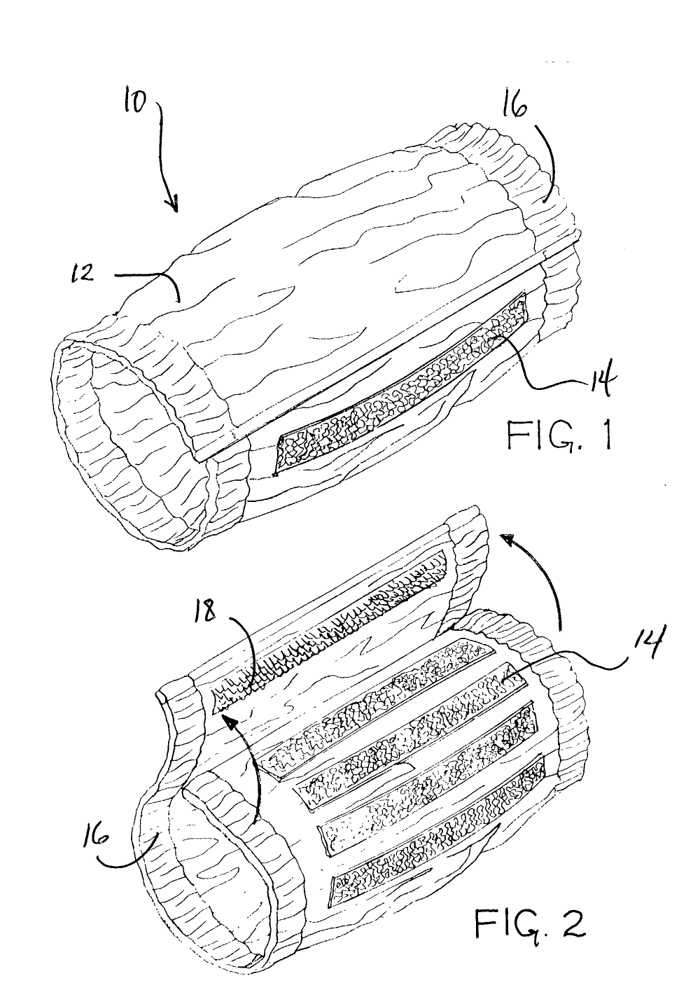

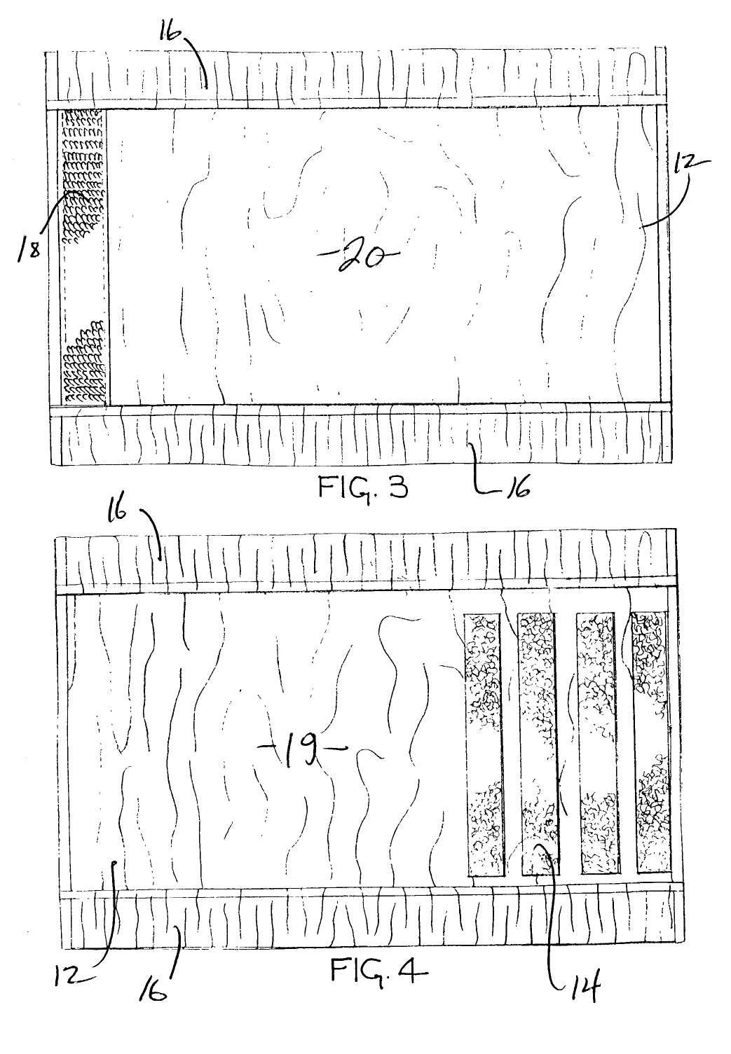

### DETAILED DESCRIPTION OF A PREFERRED EMBODIMENT

Referring to the accompanying drawings, it will be seen that a preferred embodiment of a therapeutic wrap 10 comprises a terry cloth fabric member 12 having a plurality of parallel Velcro strips 14 on the outside surface 19 and a single mating Velcro strip 18 on the inside surface 20. The length of the cloth 12 has a pair of folded margins 16 in each of which there is a stitched expandable band 15.

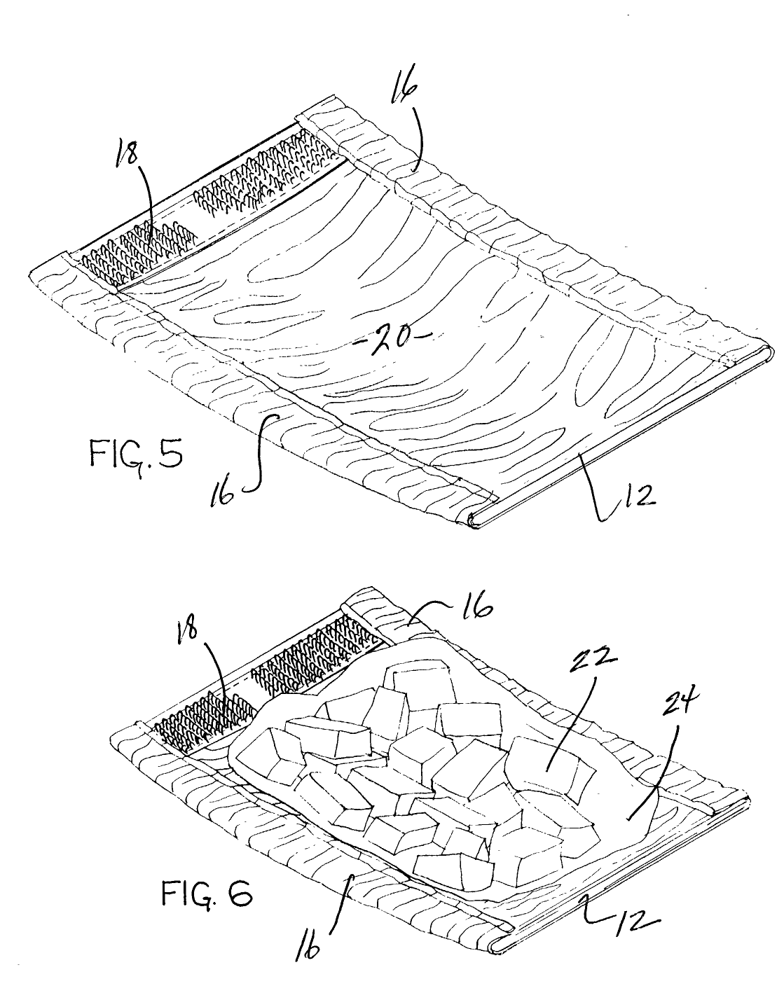

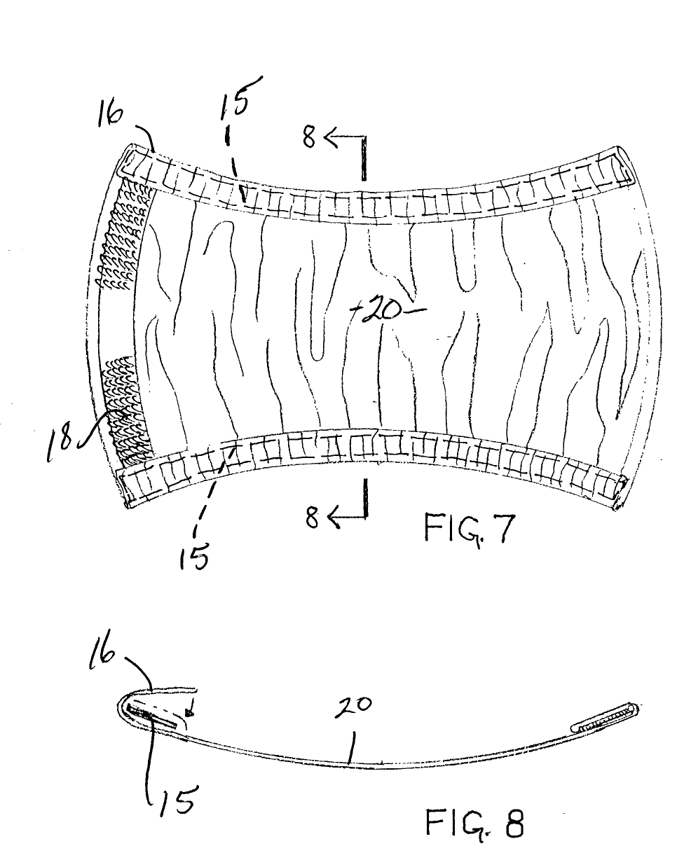

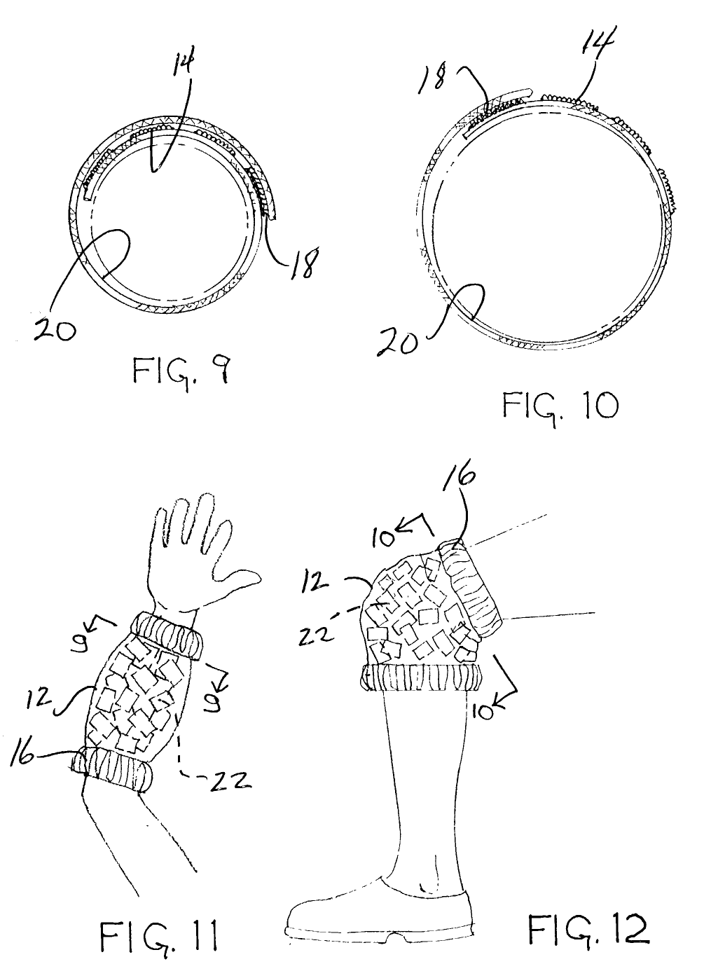

As seen best in FIGS. 5-8, inside surface 20 is configured to be slightly concave or non-planar to form a pocket for receiving a plurality of ice cubes 22 which, as shown in FIG. 6, may be provided on an optional plastic liner 24. As seen best in FIGS. 9-12, when wrap 10 is secured around an arm or knee, for example, concave inside surface 20 forms an annular pocket for securing the ice cubs 22 to the body part being treated. Moreover, margins 16 and the expandable bands 15 form an elastic seal at the ends of the annular pocket to retain the ice cubes and minimize water leakage as the ice cubes melt.

Thus it will be understood that the present invention provides a simple, low cost, yet effective, adjustable therapeutic wrap which is comfortable to wear on a limb or around a joint while containing a plurality of ice cubes or other cooling medium. It will also be understood that although a particular embodiment has been described in detail, the invention is not limited thereto, but only by the scope of the appended claims and their equivalents.

I claim:

1. A therapeutic wrap comprising:
a substantially rectangular fabric member, said member having an inside surface and an outside surface;
fastening elements affixed to said inside and outside surfaces for adjustably attaching said member to a limb;
said inside surface being substantially concave and forming a non-planar area for receiving a cooling or heating medium to be applied to said limb.

2. The therapeutic wrap recited in claim 1 further comprising folded margins along two opposed edges of said member and a respective expandable band retained within each said margin for selective expansion and contraction of said member along said margins.

3. The therapeutic wrap recited in claim 2 wherein said margins and expandable bands form seals for substantially trapping said cooling or heating medium within said pocket on said limb.

4. The therapeutic wrap recited in claim 1 wherein said cooling medium comprises ice cubes.

5. The therapeutic wrap recited in claim 1 wherein said fabric comprises terry cloth.

6. The therapeutic wrap recited in claim 1 wherein said fastening elements comprise mating hook and loop fasteners.

7. The therapeutic wrap recited in claim 6 wherein mating hook and loop fasteners are Velcro fasteners.

8. The therapeutic wrap recited in claim 1 wherein said non-planar area is configured as an open pocket.

9. The therapeutic wrap recited in claim 4 further comprising a plastic liner for receiving said ice cubes in said non-planar area.

10. The therapeutic wrap recited in claim 2 wherein said folded margins are sewn to said inside surface and wherein each said expandable band is affixed at discrete locations within a respective margin.

11. A therapeutic wrap for encircling a limb or joint for securing a cooling or heating medium in contact with the limb or joint, the wrap comprising: a wrapable member having fastening elements for adjustably attaching said member to said joint or limb;

12. The therapeutic wrap recited in claim 11 wherein said cooling medium comprises ice cubes.

13. The therapeutic wrap recited in claim 11 wherein said fastening elements comprise mating hook and loop fasteners.

14. The therapeutic wrap recited in claim 13 wherein mating hook and loop fasteners are Velcro fasteners.

15. The therapeutic wrap recited in claim 11 further comprising a plastic liner for receiving said ice cubes in said non-planar area.

16. The therapeutic wrap recited in claim 11 wherein said wrapable member comprises a fabric.

17. The therapeutic wrap recited in claim 16 wherein said fabric is terry cloth.
